(19) United States
(12) Patent Application Publication
(10) Pub. No.: US 2007/0244369 A1
(43) Pub. Date: Oct. 18, 2007

(54) Medical Imaging System for Mapping a Structure in a Patient's Body

(76) Inventors: Olivier Gerard, Viroflay (FR); Raoul Florent, Ville D'Avray (FR)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(21) Appl. No.: 11/568,915

(22) PCT Filed: May 13, 2005
(86) PCT No.: PCT/IB05/51575

§ 371 Date: Jul. 6, 2007

(30) Foreign Application Priority Data
May 17, 2004 (EP) 04300283.1

(51) Int. Cl. A61B 19/00; G06T 17/00

(52) U.S. Cl. 600/300

(57) ABSTRACT

The present invention relates to a medical imaging system for guiding a medical instrument (4) which performs a plurality of actions at a plurality of points (P₁, P₂, . . . P_{M}) in contact with a structure (3) of a body of a subject. Such a medical system comprises acquisition means for acquiring a plurality of three-dimensional (3D) image data sets (3DIS(t₁), 3DIS(t₂) . . . 3DIS(t)) of said structure (3), means (9) for associating one of said plurality of points (Pⱼ) with one of said plurality of 3D image data sets (3DIS(tᵢ)), means (10) for computing a reference 2D image data set (3DIS(t_{R})) from said plurality of 3D image data sets, means (11) for defining a transformation (TR(tᵢ)) to the points (Pⱼ) of said plurality of points which are associated with said one of said 3D image data sets (3DIS(tᵢ)) and means (13) for visualizing said transformed points (TR(tᵢ)Pⱼ).

### FIELD OF THE INVENTION

The present invention relates to a medical imaging system for mapping a structure of a patient's body using a medical instrument and three-dimensional imaging. The present invention also relates to a method to be used in said medical imaging system.

Such an invention is used for guiding the placement and operation of an invasive medical instrument in a body organ, in particular the heart.

### BACKGROUND OF THE INVENTION

Clinical applications in which an invasive medical instrument has to be guided into the body of a patient are becoming widespread. Notably the growing interest in minimally invasive methods for the treatment of cardiac diseases necessitates the development of methods and devices allowing the physician to guide a medical instrument to predetermined positions inside or outside the heart. In electrophysiology for example, it is necessary to guide a catheter to a plurality of positions on the ventricular or atrial walls in order to measure an electrical pulse or to burn wall tissues.

A method and system for mapping a structure in the body of a patient is disclosed in the European Patent Application published with publication number EP 1182619A2. A three-dimensional image data set of the structure is captured. A 3D geometrical map of the structure is generated using a medical instrument inserted into the structure in the following way: the medical instrument, which is equipped with a position sensor, is brought into contact with the structure at a multiplicity of locations on the structure, which are recorded on the 3D geometrical map. The 3D image data set is registered with the map, such that each of a plurality of image points in the 3D image data set is associated with a corresponding point in the 3D geometrical map. The 3D geometrical map is displayed such that diagnostic information directly coming or derived from the 3D image data set, for example related to blood flow in the structure, is displayed at the corresponding map point.

Such a method provides a solution for generating a 3D geometrical frame model of the structure from the locations provided by the medical instrument, in which the diagnostic information provided by the 3D image data set could be mapped. The locations of the medical instrument have to be chosen such that a geometrical shape of the structure can be built up. Based on the combined diagnostic and geometrical information, a user operating the catheter is able to identify and visualize areas of the structure, for example the heart, that are in need of treatment. A drawback of such a method is that it does not take into account the fact that the structure may have moved between two successive measurements of the medical instrument. Therefore, the obtained 3D map is not accurate.

### SUMMARY OF THE INVENTION

It is an object of the invention to provide a system which generates a 3D map of a structure of a patient's body, which is more accurate.

This is achieved by a medical imaging system comprising:
acquisition means for acquiring a plurality of three-dimensional (3D) image data sets of a structure of a body of a subject,
a medical instrument for performing a plurality of actions at a plurality of points in contact with said structure,
means for associating one of said plurality of points with one of said plurality of 3D image data sets,
means for computing a reference 3D image data set from said plurality of 3D image data sets,
means for defining a transformation for matching said one of said plurality of 3D image data sets with said reference 3D image data set,
means for applying said matching transformation to the points of said plurality of points which are associated with said one of said 3D image data sets,
means for visualizing said transformed points.

With the invention, the structure of the body, for instance a heart cavity, is explored from the inside using the medical instrument placed inside the structure, and from the outside using the 3D image acquisition means.

The acquisition means are adapted to successively acquire a plurality of 3D image data sets of said structure, for example 3D ultrasound image data sets using an ultrasound probe. It should be noted that imaging modalities other than ultrasound, such as CT or X-ray, may be used as well. An advantage of ultrasound imaging is that it shows the structure wall and vascularities. An advantage of acquiring a plurality of 3D image data sets is that they show an evolution of the structure in time. As a matter of fact, a structure of the body like, for instance, the heart is expected to move and change shape due to contractions during the cardiac cycle.

The medical instrument is adapted to perform a plurality of actions, for instance measuring an electrical activity or burning a tissue at a plurality of location points of the structure wall with which it is brought into contact. In the first case, the objective is to completely and uniformly map the structure wall. In the second case, the objective is to precisely reach desired points of the structure wall. These actions are performed successively by the medical instrument within a certain period of time.

The associating means are intended to associate a point with a 3D image data set. Advantageously, a point corresponding to an action performed at time t is associated with a 3D image data set acquired at the same time instant or at a time instant which is very close to time t. An advantage is that the associated 3D image data set provides information about the background of the structure at the instant when the action has been performed by the medical instrument.

The means for computing a reference 3D image data set from said plurality of 3D image data sets are intended to derive a reference 3D image data set, for instance, from a combination of the last two acquired 3D image data sets. Said reference 3D image data set can simply be chosen as a 3D image data set from the plurality of 3D image data sets as well.

For each 3D image data set, which has been associated with at least one point, a transformation is defined for matching said 3D image data set with said reference image data set. Such an operation is repeated for the points which are associated with another 3D image data set, using another transformation. In this way, these transformed points are registered with respect to the reference 3D image data set.

The visualization means are then adapted to provide a visualization of the transformed points, thereby forming a map of the structure. Such a map comprises at each action point a result of the action performed, for example, a measure or an indication that the tissue has been burnt.

Therefore, the map obtained with the invention is more accurate, because an adapted transformation has been applied to each point, which compensates for any deformation or motion undergone by the structure in the time between the acquisition of the reference image data set and the acquisition of the associated 3D image data set.

Advantageously, the means for visualizing said transformed points comprise sub-means for generating a representation, in which the transformed points are superimposed either with the reference 3D image data set or with the current 3D image data set acquired at time t after transformation by the matching transformation defined for the current 3D image data set. A first advantage is that such a superimposition may help the user to place the action points in relation to the surrounding anatomy. Another advantage is that said representation may help the user to decide where to perform a next action.

In a first embodiment of the invention, the reference image data set is chosen as a fixed 3D image data set, for instance acquired at a time t₁. In other words, a fixed map is generated and each new point is registered with respect to said fixed reference. An advantage is that reading of the map is facilitated because, when a new point appears on the map, the points which have been previously processed remain unchanged.

In a second embodiment of the invention, the reference image data set is chosen as a current 3D image data set acquired at a current time t. In this case, an up-to-date map is obtained, which moves with the structure. An advantage is that at the current time t the visualized map corresponds to the real state of the structure in the body. The generated map is also more realistic, because it moves with the structure.

In a third embodiment of the invention, a geometrical transformation is applied to the reference 3D image data set. The objective is for example to ensure that the structure and consequently the map is visualized in a given orientation, which is familiar to the user. An advantage is that such a geometrically transformed map can be more easily interpreted by the user.

In a fourth embodiment of the invention, the visualization means are adapted to provide a view of a region of interest of the medical instrument. A first advantage of this fourth embodiment of the invention is that it provides a zoom-in of the vicinity of the medical, instrument, which improves the visualization of the region of interest. A second advantage is that such a view provides another perspective of the structure. Therefore, combined with the representation, such a view may help the user to define a next location for performing an action with the medical instrument in a quicker and more efficient way.

These and other aspects of the invention are apparent from and will be elucidated with reference to the embodiments described hereinafter.

### BRIEF DESCRIPTION OF THE DRAWINGS

The present invention will now be described in more detail, by way of example, with reference to the accompanying drawings, wherein:

FIG. 1 is a schematic drawing of a system in accordance with the invention,

FIG. 2 is a schematic drawing of the association means in accordance with the invention,

FIG. 3 is a schematic drawing of the means for localizing a 3D image data set in accordance with the invention,

FIG. 4 is a schematic drawing of the means for defining a transformation in accordance with the invention,

FIG. 5 is a schematic drawing of the means for applying the transformation defined for a 3D image data set to the points associated with said 3D image data set in accordance with the invention,

FIG. 6 is a schematic drawing of a map provided by the visualization means in accordance with the invention,

FIG. 7 is a schematic drawing of a representation in which the transformed points are superimposed with the reference 3D image data set in accordance with a first embodiment of the invention,

FIG. 8 is a schematic drawing of a representation in which the transformed points are superimposed with the reference 3D image data set in accordance with a second embodiment of the invention,

FIG. 9 is a schematic drawing of the means for applying a geometrical transformation to the reference 3D image data set in accordance with a third embodiment of the invention,

FIG. 10 is a schematic drawing of a view of a region of interest of the medical instrument provided by the visualization means in accordance with a fourth embodiment of the invention,

FIG. 11 is a schematic representation of a method in accordance with the invention.

### DETAILED DESCRIPTION OF THE INVENTION

The present invention relates to a system for mapping a structure of a patient's body using a medical instrument and three-dimensional imaging. In the following, the system in accordance with the invention will be described in more detail by means of, in this case, the application of an electrophysiology catheter introduced into a heart cavity, for instance the left ventricular or the right atrium chamber, in order to measure an electrical activity of the heart or to burn diseased tissues.

However, the invention is not limited to electrophysiology procedures and can more generally be used for guiding any other medical instrument in the patient's body, like for instance a needle.

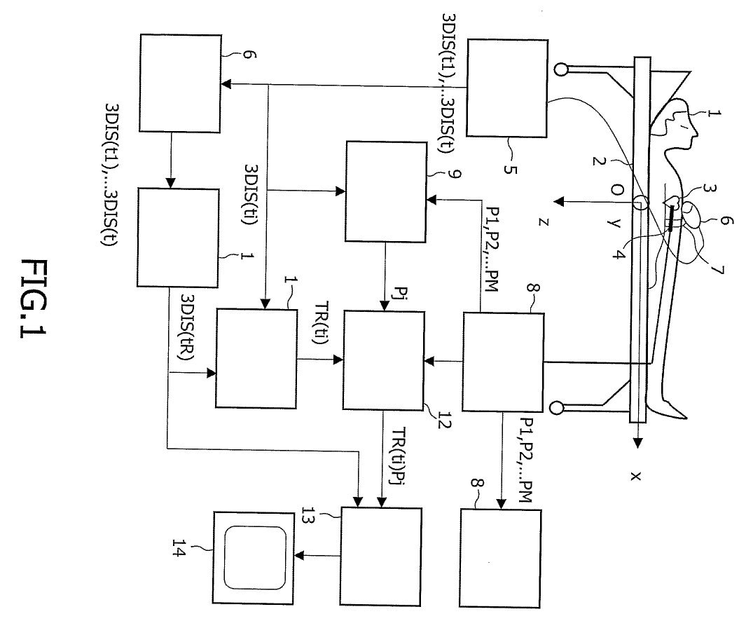

The schematic drawing of FIG. 1 shows a patient 1, who is arranged on a patient table 2 and whose symbolically indicated heart 3 is subjected to a treatment by means of a catheter 4 introduced into the body. The system in accordance with the invention comprises means 5 for acquiring a plurality of 3D image data sets of the structure 3DIS(t₁), 3DIS(t₂), . . . ,3DIS(t). In the following, the plurality of 3D image data sets is a plurality of ultrasound image data sets acquired from an ultrasound probe 6, which has been placed on the patient's body and fixed by fixation means, for instance a belt 7 or a stereotactic arm. It should be noted however that the invention is not limited to ultrasound acquisition means and that CT, MRI or X-Ray acquisition means could be used as well.

Advantageously, the 3D acquisition means 5 are adapted to provide a live 3D image data set. For instance, the 3D image data sets 3DIS(t₁), 3DIS(t₂), . . . ,3DIS(t) are acquired at predetermined phases of the cardiac cycle. It should be noted however that they can be acquired at any phase of the cardiac cycle as well.

The plurality of 3D image data sets is stored in a memory 6.

The system in accordance with the invention comprises a medical instrument 4 to be guided inside the structure 3 for performing a plurality of actions at a plurality of location points P₁, P₂, . . . , P_{M}, where M is an integer, in contact with said structure. Said plurality of actions is controlled by a controller 8 and the results of this plurality of actions are stored in a memory 8. The system in accordance with the invention further comprises means 9 for associating one of said plurality of points Pⱼ with one of said plurality of 3D image data sets 3DIS(tᵢ), means 10 for computing a reference 3D image data set 3DIS(t_{R}) from said plurality of 3D image data sets, means 11 for defining a transformation TR(tᵢ) for matching said one of said plurality of 3D image data sets 3DIS(tᵢ) with the reference 3D image data set 3DIS(t_{R}), means 12 for applying said matching transformation TR(tᵢ) to the points Pⱼ of said plurality of points which are associated with said one of said 3D image data sets 3DIS(tᵢ) and means 13 for visualizing said transformed points TR(tᵢ)Pⱼ using display means 14.

In accordance with the invention, the medical instrument 4 has an extremity, which is adapted to perform an action Aⱼ, such as measuring an electrical activity or burning a tissue, when it is brought into contact with a location point Pⱼ of the inside wall of the structure. In the particular case of an electrophysiology procedure, this extremity of the catheter 4 is called a tip. Advantageously, the controller 8 comprises sub-means for localizing the tip of the catheter, which give the precise location of the location point contacted by the medical instrument. In a first alternative, the medical instrument 4 is equipped with an active localizer, for instance an RF coil, as described above for localizing the ultrasound probe 6. Said tip is a small and thin segment, which is very echogenic and leaves a specific signature in the 3D ultrasound image data set. In a second alternative, the tip localization sub-means advantageously employ image processing techniques, which are well known to those skilled in the art, for enhancing either a highly contrasted blob or an elongated shape in a relatively uniform background.

Therefore, the tip localization sub-means are adapted to provide a location of a contact point Pⱼ=(xⱼ, yⱼ, zⱼ) of the medical instrument 4 and the inside wall of the structure 3 at a time tⱼ. In the first alternative, such a location is directly expressed in a fixed referential, for instance a referential (O, x, y, z) of the clinical intervention room. In the second alternative, it is firstly expressed in a local referential (O′, x′, y′, z′) of the ultrasound probe 6 and converted into coordinates within the referential of the clinical intervention room (O, x, y, z) by conversion means, well-known to those skilled in the art.

The system comprises means 9 for associating a location point Pⱼ, j being an integer, with one 3D image data set from the plurality of 3D image data sets 3DIS(t₁), 3DIS(t₂), . . . ,3DIS(t). Advantageously, the location point Pⱼ, which corresponds to an action Aⱼ performed at a time instant tⱼ, is associated with the 3D image data set 3DIS(tᵢ) acquired at time tᵢ, said time being the closest time to tⱼ among the times of acquisition of the 3D image data sets 3DIS(t₁) to 3DIS(t).

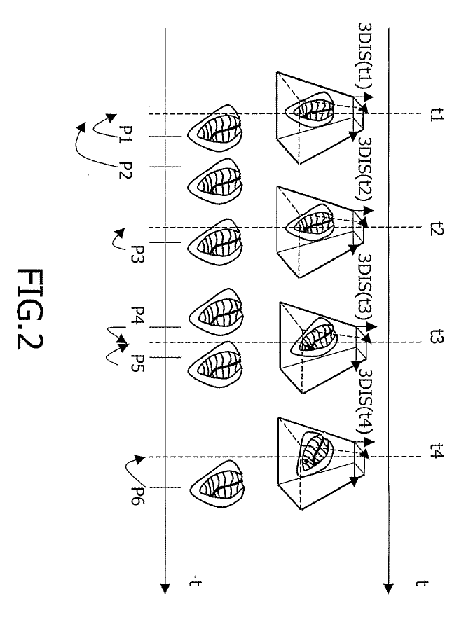

Referring to FIG. 2, the location points P₁, P₂ are therefore associated with the 3D image data set 3DIS(t₁), the location point P₃ with the 3D image data set 3DIS(t₂), the location points P₄, P₅ with the 3D image data set 3DIS(t₃) and the location point P₆ with the 3D image data set 3DIS(t₄).

An advantage is that the associated 3D image data set 3DIS(tᵢ) can be considered to represent a state of the structure 3 at the instant tⱼ at which the action was performed at the location point Pⱼ. It should be noted that more than one location may be associated with one 3D image data set.

The means 10 are intended to derive a reference 3D image data set 3DIS(t_{R}). For instance, the reference 3D image data set 3DIS(t_{R}) is built up by combining the last two acquired 3D image data sets 3DIS(t−1) and 3DIS(t), especially if there is a location point acquired at a time close to t−½. Said reference 3D image data set can also simply be chosen as a 3D image data set from the plurality of 3D image data sets, for instance as the first 3D image data set 3DIS(t₁) acquired at a time t₁ or the current 3D image data set 3DIS(t) acquired at a time t. In the following, it is assumed that the reference 3D image data set 3DIS(t_{R}) has been acquired at a time t_{R}, where R is an integer that is a priori different from i. Between time t_{R} and time tᵢ, both the ultrasound probe 6 and the structure 3 may have moved.

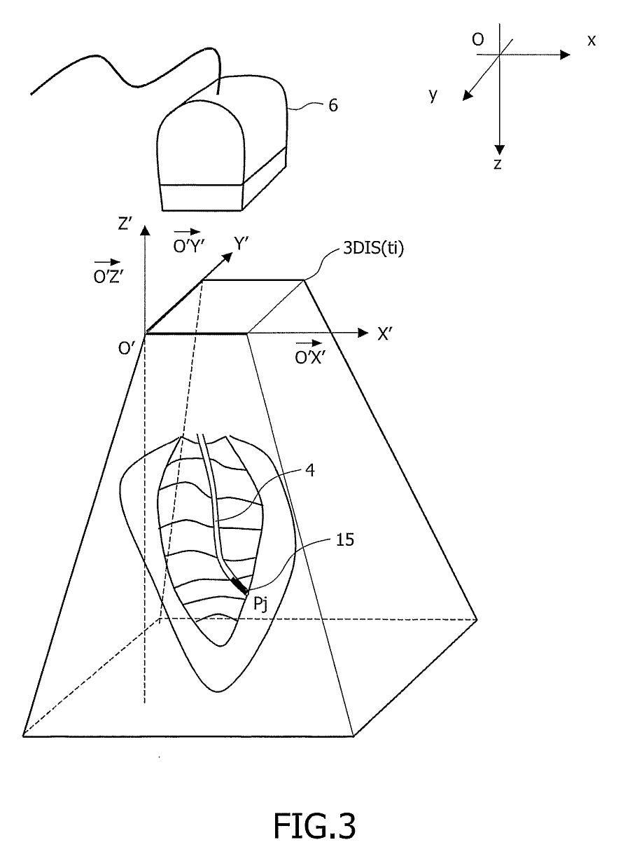

Referring to FIG. 3, the system further comprises means 11 for defining a transformation TR(tᵢ) which matches the 3D image data set 3DIS(tᵢ) with the reference 3D image data set 3DIS(t_{R}) in the fixed referential (O, x, y, z) of the clinical intervention room.

Referring to FIG. 3, the system in accordance with the invention advantageously comprises means for localizing the ultrasound probe 6 in a fixed referential of coordinates with respect to the ultrasound probe, for instance the referential of coordinates (O, x, y, z) of the clinical intervention room.

Such a localization is for instance based on an active localizer, well-known to those skilled in the art, which is arranged on the ultrasound probe 6. Said active localizer, for instance an RF coil, is intended to transmit an RF signal to an RF receiving unit placed under the patient's body and for instance integrated into the table 2. The RF receiving unit transmits the received signal to measuring means for measuring a position of the ultrasound probe 6 in the referential (O, x, y, z) of the clinical intervention room. It should be noted that the active localizer must provide a precise measurement of the position and of the orientation of the ultrasound probe 6. It should further be noted that a led-based optical localizer could be used as well. A first advantage of such a localization is that it is very precise. A second advantage is that it is performed in real-time and therefore can be triggered during the clinical procedure, if necessary.

As already mentioned, the ultrasound probe 6 is likely to move during the clinical intervention due to external movements of the patient, such as respiratory movements. Therefore, means for localizing the ultrasound probe 6 are intended to provide a localization of the ultrasound probe 6 at a time tᵢ, which simultaneously gives a localization of the 3D image data set acquired at time tᵢ in the referential of coordinates (O, x, y, z). Such a localization completely defines a position and orientation of the ultrasound probe 6 and the 3D image data set 3DIS(tᵢ) within the referential (O, x, y, z) and for instance comprises the coordinates of a point O′ and of three orthogonal vectors \vec{O′X′}, \vec{O′Y′}, \vec{O′Z′}.

Advantageously, a local referential of coordinates (O′, x′, y′, z′)(t) is attached to the ultrasound probe 6 at time t. Such a referential (O′, x′, y′, z′)(t) is particularly useful in order to localize structures of interest in the 3D image data set, such as the medical instrument 4 or the structure 3. Such a local referential moves with the 3D image data set. Therefore, a localization Loc(tᵢ) of the local referential (O′, x′, y′, z′)(tᵢ) attached to the 3D image data set 3DIS(tᵢ) and a localization Loc(t_{R}) of the local referential (O′, x′, y′, z′)(t_{R}) attached to the reference 3D image data set 3DIS(t_{R}) are provided within the referential (O, x, y, z) of the clinical intervention room. Consequently, a first transformation Tr(tᵢ), which matches the localizations Loc(tᵢ) and Loc(t_{R}) can be defined within the referential (O, x, y, z) by the means 11 for defining a transformation.

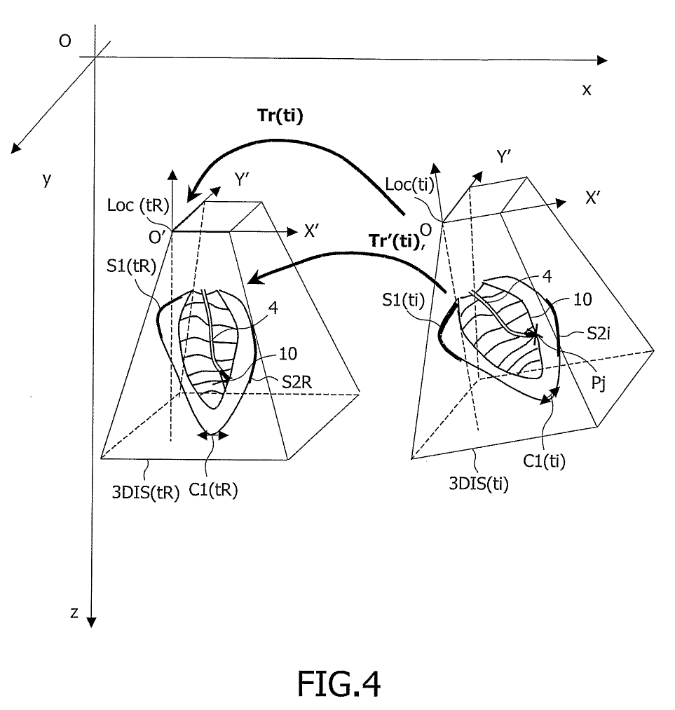

Advantageously, the means 11 for defining a transformation TR(tᵢ), which matches the 3D image data set 3DIS(tᵢ) with the reference image data set 3DIS(t_{R}), comprises sub-means for segmenting the structure 3 both within the local referentials (O, x′, y′, z′)(tᵢ) and (O, x′, y′, z′)( t_{R}) of the ultrasound probe 6. Referring to FIG. 4, said sub-means are adapted to segment a first surface S1 (tᵢ) of said structure in the 3D image data set 3DIS(tᵢ) and a second surface S₁(t_{R}) of said structure in the reference 3D image data set 3DIS(t_{R}). Given a set of points of said first surface S₁(tᵢ), a corresponding second set of points may be searched for use in the reference 3D image data set 3DIS(t_{R}) using, for instance, an Iterative Closest Point Algorithm, well known to those skilled in the art. The means for defining a transformation TR(tᵢ) are adapted to seek a second transformation Tr′(tᵢ), for instance from a family of transformations, that minimizes a mean square error between the first and second sets of points S₁(tᵢ), S₁(t_{R}). Advantageously, additional features like curvature measurements C₁(tᵢ), C₁(t_{R}) may be used to improve the matching. The second transformation Tr′(tᵢ) is then applied to all the points of the first surface S₁(tᵢ).

It should be noted that the medical instrument 4 must not interfere in such a process of finding a matching transformation, since the medical instrument may have moved with respect to the structure 3.

Therefore, referring to FIG. 4, the transformation TR(tᵢ) may be decomposed into a first transformation Tr(tᵢ), which matches the localization Loc(tᵢ) of the local referential (O′, x′, y′, z′)(tᵢ) of the ultrasound probe 6 within the referential (O, x, y, z) at time ti with the localization Loc(t_{R}) of the local referential (O′, x′, y′, z′)(t_{R}) of the ultrasound probe 6 at time t_{R}, and into a second transformation Tr′(tᵢ), which matches the structure 3 within the 3D image data set 3DIS(tᵢ) with the structure 3 within the reference 3D image data set 3DIS(t_{R}).

In this way, an adapted transformation TR(tᵢ) is defined for each 3D image data set 3DIS(tᵢ) which has location points Pⱼ associated with. Therefore, a plurality of transformations is defined during the clinical intervention.

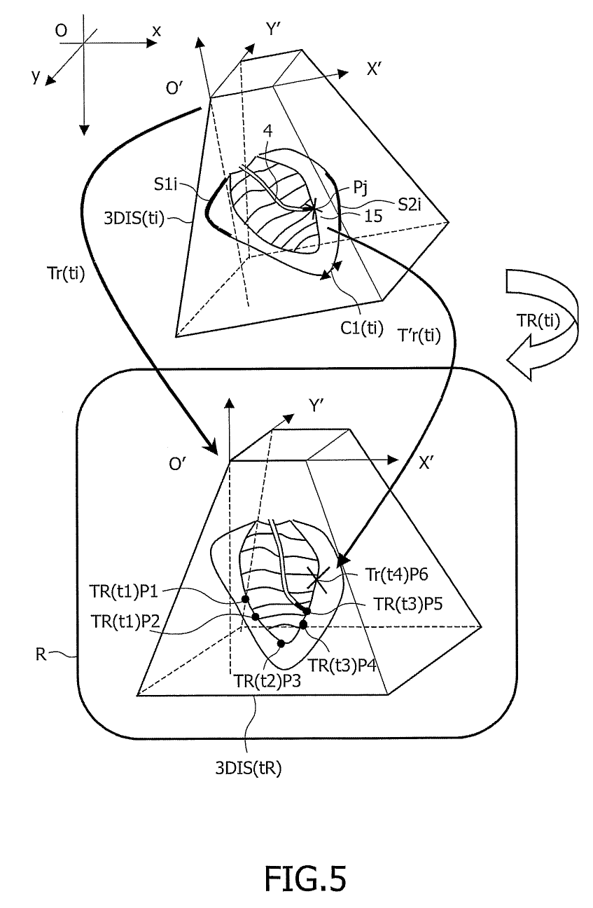

Referring to FIG. 5, the defined transformation TR(tᵢ) is then applied by means 12 to the location point(s) Pⱼ associated with the 3D image data set 3DIS(tⱼ₎. In this way, a transformed location point TR(tᵢ)Pⱼ is obtained, which is registered with respect to the reference image data set 3DIS(t_{R}).

The system in accordance with the invention finally comprises means 13 for visualizing the plurality of transformed location points TR(tᵢ)Pⱼ, obtained by applying said plurality of transformations.

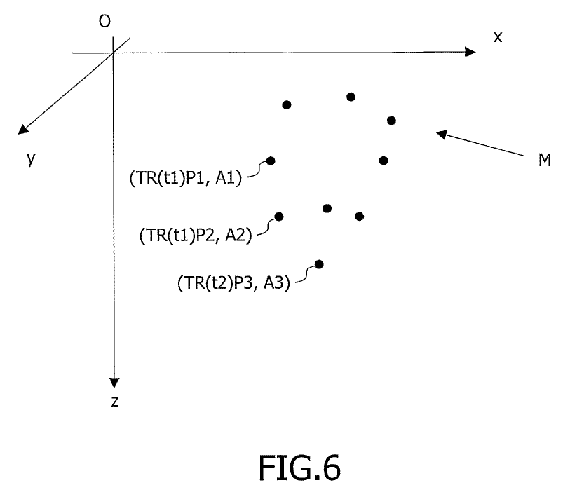

Referring to FIG. 6, the plurality of transformed location points forms a map M of the structure 3 in which the result of the action Aⱼ, for example a measurement value or an indication that the tissue has been burnt, is given at each transformed location point TR(tᵢ)Pⱼ. Said map is registered with respect to the reference 3D image data set 3DIS(t_{R}), because the plurality of location points P₁, P₂, . . . , P_{M} which constitutes this map has been registered by adapted transformations with respect to this reference image data set.

The map M is displayed by display means 14.

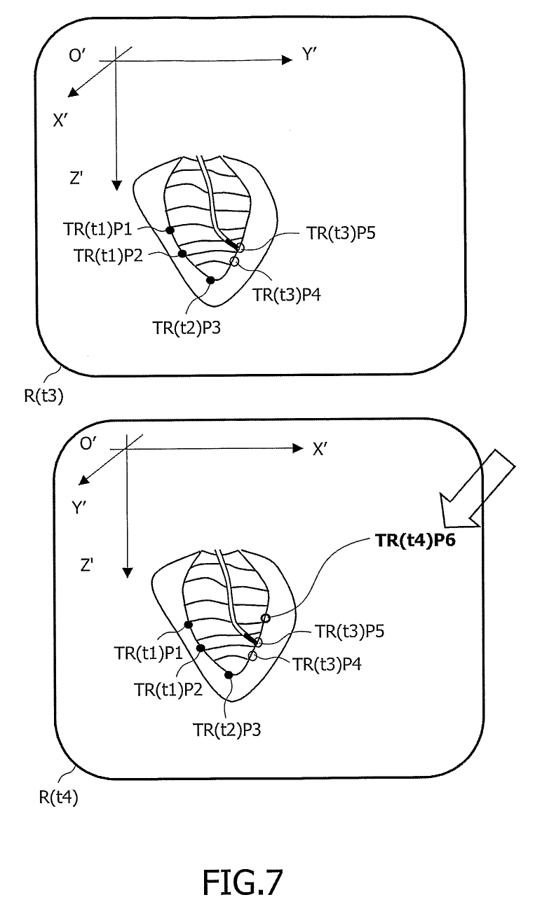

In a first embodiment of the invention, shown in FIG. 7, the reference 3D image data set TR(tᵢ)Pⱼ is a fixed 3D image data set, for example the first 3D image data set 3DIS(tᵢ) acquired at time t₁. Therefore, a location point Pⱼ associated with a 3D image data set 3DIS(tᵢ) is firstly transformed into a transformed point TR(tᵢ)Pⱼ by the transformation TR(tᵢ), which matches the 3D image data set 3DIS(tᵢ) with the reference 3D image data set 3DIS(t_{R}), and then visualized by the visualization means 13.

In a first alternative, the means 13 for visualizing said transformed points comprise sub-means for generating a representation R, in which said transformed points TR(tᵢ)Pⱼ are superimposed with said reference 3D image data set 3DIS(t_{R}), as shown in FIG. 7.

Therefore, the representation R provided by the visualization means 13 comprises a fixed anatomical background on which the transformed location points TR(tᵢ)Pⱼ are successively superimposed. It should be noted that, as shown in FIG. 7, the position of the medical instrument is a priori not updated.

Advantageously, the system further comprises means for excluding the medical instrument 4 from the reference 3D image data set, for instance by using detection means based on image processing techniques which are well-known to those skilled in the art.

A first advantage of such a representation is that it is obtained in a simple way, because a single transformation TR(tᵢ) is applied to each location point Pⱼ. A second advantage is that reading of the representation is facilitated because, when a new location point appears on the representation R, the points which have been previously processed remain unchanged.

In a second alternative, the medical imaging system in accordance with the invention further comprises means for applying said transformation TR(tᵢ) to the 3D image data set 3DIS(tᵢ). A transformed 3D image data set TR(tᵢ)3DIS(tᵢ) is obtained, which is used to generate the representation R(tᵢ) at time tᵢ. Therefore, at time ti the representation R(tᵢ) shows the currently transformed location points TR(tᵢ)Pⱼ and the previously transformed points superimposed with the 3D image data set TR(tᵢ)(3DIS(tᵢ)). A first advantage is that in the representation R both the medical instrument 4 and the structure 3 are updated. Therefore, the anatomical background formed by the image data is up to date. Moreover, by applying the transformation TR(tᵢ) to the 3D image data set 3DIS(tᵢ), compensation is provided for any motion of the structure 3 with respect to the reference 3D image data set 3DIS(t_{R}). Consequently, a second advantage is that the guiding of the medical instrument to a next target point is facilitated.

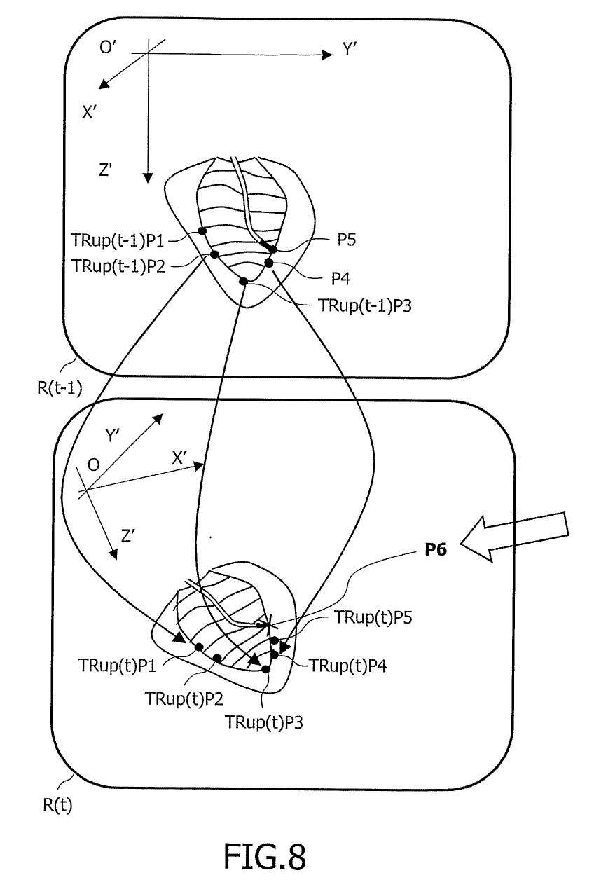

In a second embodiment of the invention, shown in FIG. 8, the reference image data set 3DIS(t_{R}) is chosen as a 3D image data set acquired at a current time t, for example, as the current 3D image data set. In this case, an up-to-date representation R(t) is obtained, which moves with the structure 3. In this case, a new location point Pⱼ associated with the 3D image data set 3DIS(t) is superimposed without any transformation to the reference 3D image data set 3DIS_{R}(t), because it corresponds to the current 3D image data set with which it is associated. In this case, all the previously acquired location points which have been superimposed to the previous reference 3D image data set 3DIS(t−1) in order to form the representation R(t−1) at time t−1 need to be registered with respect to the reference 3D image data set 3DIS(t) at time t so that the representation can be updated.

In a first alternative, referring to FIG. 8, the previously acquired location points superimposed with the previous reference 3D image data set 3DIS(t−1) are all transformed, by an identical update transformation TRᵤₚ(t) which matches the reference 3D image data set 3DIS(t−1) at time t−1 with the reference 3D image data set 3DIS(t) at time t, into transformed points TRᵤₚ(t)P₁, TRᵤₚ(t)P₂, TRᵤₚ(t)P₃, TRᵤₚ(t)P₄ and TRᵤₚ(t)P₅.

Consequently, in accordance with the second embodiment of the invention, a location point Pⱼ acquired at time tⱼ is transformed by a global transformation TR(tᵢ), which comprises a succession of successive update transformations TRᵤₚ at times t−1, t, t+1. Therefore, at time t, the point Pj acquired at time t−1 is transformed into a transformed point Trᵤₚ(t)Pj, which is further transformed at time t+1 by an update transformation TRᵤₚ(t+1) etc.

A first advantage is that at the current time t the visualized representation R(t) corresponds to the live state of the structure in the body. The generated map of the location points is also more realistic. A second advantage is that the computation needs are reasonable.

In a second alternative, a location point Pⱼ acquired at time tⱼ and associated with the 3D image data set 3DIS(tᵢ) is successively transformed by a plurality of transformations TRᵢ(tⱼ₊₁), . . . , TRᵢ(t). The location point Pⱼ is transformed at time t into a transformed point TRᵢ(t)Pⱼ by a transformation TRᵢ(t), which registers the 3D image data set 3DIS(tᵢ) with the reference 3D image data set 3DIS(t); the same location point Pⱼ is further transformed at time t+1 into a transformed point TRᵢ(t+1)Pⱼ by a transformation TRᵢ(t+1) which registers the 3D image data set 3DIS(tᵢ) with the reference 3D image data set 3DIS(t+1) etc. An advantage is that errors due to successive transformations do not accumulate.

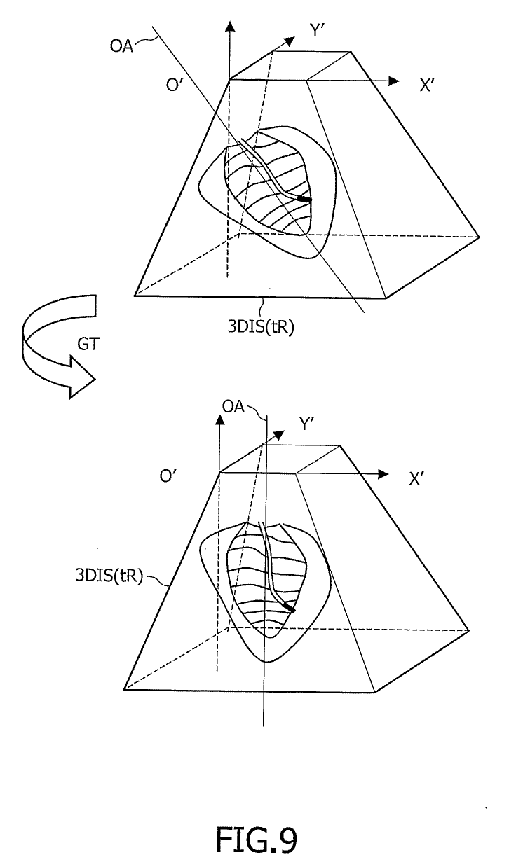

In a third embodiment of the invention, the reference image data set 3DIS(t_{R}) is transformed by a geometrical transformation. The objective of such a geometrical transformation is for example to ensure that the structure and consequently the representation is visualized by the user in a way he is familiar with. For example, such a geometrical transformation may place the structure in the center of the 3D image data set or put it in a desired orientation. Referring to FIG. 9, an orientation axis OA of the structure 3 may be detected in the reference 3D image data set 3DIS(t_{R}) by using image processing techniques known to those skilled in the art. A geometrical transformation GT is then defined, which, when applied to the structure 3, will place it in the desired position and orientation. Such a geometrical transformation has to be applied to the transformed location points before superimposing them to the reference 3D image data set 3DIS(t_{R}). An advantage is that such a geometrically transformed representation can be more easily interpreted by the user.

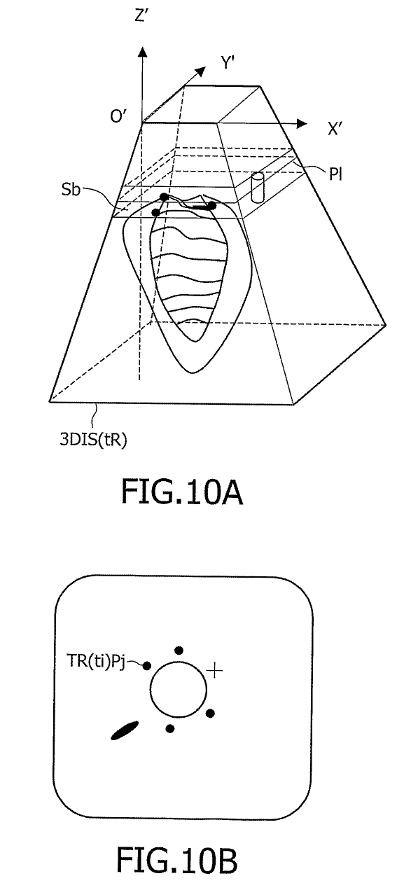

In a fourth embodiment of the invention, the visualization means 12 are adapted to provide a view of a region of interest of the medical instrument 4. Referring to FIGS. 10A and 10B, such a view is for instance generated by choosing a plane P1 in the reference 3D image data set 3DIS_{R}, which contains the tip of the medical instrument 4 and which is perpendicular to the medical instrument. This is achieved by defining a slab Sb of the 3D image data set centered on this plane P1. The visualization means 12 may advantageously comprise sub-means for generating a 3D rendered view of this slab on which the transformed location points corresponding to this region of interest are superimposed. A first advantage of this fourth embodiment of the invention is the possibility to provide a zoom-in of the vicinity of the medical instrument 4, which improves the visualization of the region of interest. A second advantage is that such a view provides another perspective of the structure 3. Therefore, combined with the representation, such a view may help the user to define a next location for performing an action with the medical instrument 4 in a quicker and more efficient way. In particular, the vicinity of the entrance of the pulmonary vein in the left atrium is a region of great interest, because it plays a role in heart diseases which require burning tissues in this region of interest. Referring to FIGS. 10A and 10B, a view of the vicinity of the pulmonary vein is very likely to help the user decide on a next location for performing an action with the medical instrument.

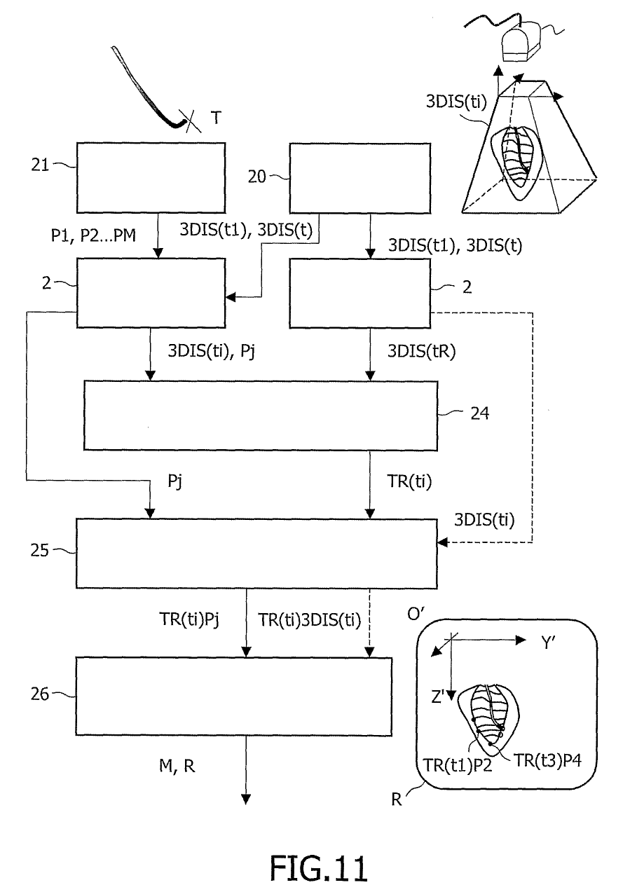

The invention also relates to a method of mapping a structure of a patient's body using a medical instrument and three-dimensional imaging. Referring to FIG. 11, such a method comprises the steps of:
acquiring 20 a plurality of three-dimensional (3D) image data sets 3DIS(t₁), 3DIS(t₂), 3DIS(t) of a structure 3 of a body of a subject,
performing 21 a plurality of actions at a plurality of points P₁, P₂, . . . P_{M} in contact with said structure,
associating 22 one of said plurality of points Pⱼ with one of said plurality of 3D image data sets 3DIS(t₁), 3DIS(t₂), . . . , 3DIS(t),
computing 23 a reference 3D image data set (3DIS(t_{R})) from said plurality of 3D image data sets (3DIS(t₁), 3DIS(t₂) . . . 3DIS(t)),
defining 24 a transformation TR(tᵢ) for matching one of said plurality of 3D image data sets 3DIS(tᵢ) with a reference 3D image data set 3DIS(t_{R}) comprised within said plurality of 3D image data sets,
applying 25 said matching transformation TR(tᵢ) to the points Pⱼ of said plurality of points P₁, P₂, . . . P_{M} which are associated with said one of said 3D image data sets 3DIS(tᵢ),
visualizing 26 said transformed points TR(tᵢ)Pⱼ.

The drawings and their description hereinbefore illustrate rather than limit the invention. It will be evident that there are numerous alternatives, which fall within the scope of the appended claims. In this respect the following closing remarks are made: there are numerous ways of implementing functions by means of items of hardware or software, or both. In this respect, the drawings are very diagrammatic, each representing only one possible embodiment of the invention. Thus, although a drawing shows different functions as different blocks, this by no means excludes that a single item of hardware or software carries out several functions, nor does it exclude that a single function is carried out by an assembly of items of hardware or software, or both.

Any reference sign in a claim should not be construed as limiting the claim. Use of the verb “to comprise” and its conjugations does not exclude the presence of elements or steps other than those stated in a claim. Use of the article “a” or “an” preceding an element or step does not exclude the presence of a plurality of such elements or steps.

1. A medical imaging system comprising:
acquisition means for acquiring a plurality of three-dimensional (3D) image data sets (3DIS(t₁), 3DIS(t₂) . . . 3DIS(t)) of a structure (3) of a body of a subject at times t₁, t₂ . . . t,
a medical instrument for performing a plurality of actions at a plurality of points (P₁, P₂, . . . P_{M}) in contact with said structure,
means for associating one of said plurality of points (Pⱼ) with one of said plurality of 3D image data sets (3DIS(tᵢ)),
means for computing a reference 3D image data set (3DIS(t_{R})) from said plurality of 3D image data sets (3DIS(t₁), 3DIS(t₂) . . . 3DIS(t)),
means for defining a transformation (TR(tᵢ)) for matching said one of said plurality of 3D image data sets (3DIS(tᵢ)) with said reference 3D image data set (3DIS(t_{R})),
means for applying said matching transformation (TR(tᵢ)) to the points (Pⱼ) of said plurality of points which are associated with said one of said plurality of said 3D image data sets (3DIS(tᵢ)),
means for visualizing said transformed points (TR(tᵢ)Pⱼ).

2. A medical imaging system as claimed in claim 1, wherein said reference image data set (3DIS(t_{R}) is a fixed 3D image data set (3DIS(t₁)).

3. A medical imaging system as claimed in claim 1, wherein said reference image data set (3DIS(t_{R})) is a current 3D image data set (3DIS(t)).

4. A medical imaging system as claimed in claim 1, wherein said means for visualizing said transformed points (TR(tᵢ)Pj) comprise sub-means for generating a representation (R), in which said transformed points are superimposed with said reference 3D image data set (3DIS(t_{R})).

5. A medical imaging system as claimed in claim 2, comprising means for applying said current matching transformation (TR(t)) to said current 3D image data set (3DIS(t)), and wherein said means for visualizing said transformed points (TR(tᵢ)Pj) comprise sub-means for generating a representation (R), in which said transformed points are superimposed with said transformed current 3D image data set (TR(3DIS(t))).

6. A medical imaging system as claimed in claim 1, wherein said means for visualizing said transformed points (TR(tᵢ)Pⱼ) comprise sub-means for generating a view of a region of interest of the medical instrument, which is perpendicular to a plane (P1) comprising an extremity of said medical instrument.

7. A medical imaging system as claimed in claim 1, comprising means for localizing said plurality of location points (P₁, P₂, . . . P_{M}) in a fixed referential of coordinates (O, x, y, z).

8. A medical imaging system as claimed in claim 1, comprising means for localizing said plurality of 3D image data sets (3DIS(t₁), 3DIS(t₂) . . . 3DIS(t)) in a fixed referential of coordinates (O, x, y, z).

9. A medical imaging system as claimed in claim 1, wherein said reference image data set (3DIS(t_{R})) has been subjected to a geometrical transformation (GT).

10. A medical imaging system as claimed in claim 8, wherein said transformation (TR(tᵢ)) comprises a first transformation (Tr(tᵢ)) for matching a local referential (O′, x′, y′, z′)(tᵢ) of said one of said plurality of 3D image data sets (3DIS(tᵢ)) with a reference local referential (O′, x′, y′, z′)(t_{R}) of said reference 3D image data set (3DIS(t_{R})) within said fixed referential of coordinates (O, x, y, z), and a second transformation (Tr′(tᵢ)) for matching a location (S1(tᵢ)) of said structure in said one of said plurality of 3D image data sets (3DIS(tᵢ)) with a reference location (S1(t_{R})) of said structure in said reference 3D image data set (3DIS(t_{R})) within said matched local referential of coordinates Tr(tᵢ)(O′, x′, y′, z′) of said 3D image data set (3DIS(tᵢ)).

11. A medical imaging system as claimed in claim 3, wherein said transformation (TR(tᵢ)) comprises successive update transformations (TRᵤₚ) for transforming a location of a transformed point (Trᵤₚ(t−1)Pⱼ) in a previous reference 3D image data set (3DIS_{R}(t−1)) acquired at time t−1 into a location TRᵤₚ(t)Pⱼ) in the current reference 3D image data set (3DIS_{R}(t)) acquired at time t.

12. A medical imaging system as claimed in claim 3, wherein a new transformation (TRᵢ(t)) for matching said one of said plurality of 3D image data sets (3DIS(tᵢ)) with said current 3D image data set (3DIS(t)) is applied to said point (Pⱼ) associated with said one of said plurality of 3D image data sets (3DIS(tᵢ)) when said reference 3D image data set (3DIS(t_{R})) is replaced by said current 3D image data set (3DIS(t)).

13. A medical imaging system as claimed in claim 1, wherein said structure is a heart and said medical instrument is an electrophysiology catheter.

14. A medical imaging system as claimed in claim 1, wherein said association means are adapted to associate a point (Pⱼ) acquired at a time tⱼ with a 3D image data set (3DIS(tᵢ)) acquired at a time tᵢ which is close to time tⱼ.

15. A medical imaging system as claimed in claim 1, wherein said acquisition means are intended to acquire a plurality of 3D ultrasound image data sets using an ultrasound probe arranged on the body of the subject.

16. A medical imaging method, comprising the steps of:
acquiring a plurality of three-dimensional (3D) image data sets (3DIS(t₁), 3DIS(t₂) . . . 3DIS(t)) of a structure of a body of a subject,
performing a plurality of actions at a plurality of location points (P₁, P₂, . . . P_{M}) in contact with said structure,
associating one of said plurality of points (Pⱼ) with one of said plurality of 3D image data sets (3DIS(tᵢ)),
computing a reference 3D image data set (3DIS(t_{R})) from said plurality of 3D image data sets (3DIS(t₁), 3DIS(t₂) . . . 3DIS(t)),
defining a transformation (TR(tᵢ)) for matching one of said plurality of 3D image data sets with said reference 3D image data set (3DIS(t_{R})),
applying said matching transformation (TR(tᵢ)) to the location points of said plurality of location points which are associated with said one of said plurality of 3D image data sets, and
visualizing said transformed points (TR(tᵢ)Pⱼ).
